(19) United States
(12) Patent Application Publication
(10) Pub. No.: US 2012/0111739 A1
(43) Pub. Date: May 10, 2012

(54) Dual Frequency Impedance Measurement of Hematocrit in Strips

(76) Inventors: John J. Pasqua, Wellington, FL (US); Christine Shields, Boca Raton, FL (US); Angela Carlson, Lake Worth, FL (US)

(21) Appl. No.: 12/247,563

(22) Filed: Oct. 8, 2008

(51) Int. Cl. G01N 33/50; G01N 27/26

(52) U.S. Cl. 205/777.5; 204/403.01; 204/403.14

(57) ABSTRACT

This invention is a method for determining a hematocrit value. The steps include applying a first pulse of potential excitation at a first frequency to a test strip containing a fluid sample. The method also includes applying a second pulse at a second frequency that is higher than the first frequency. Based on first and second impedance measurements associated with each pulse, a hematocrit value may be determined. Also, a concentration of an analyte contained within the fluid sample may be determined based on the hematocrit value.

### FIELD OF THE INVENTION

The present invention relates to the field of diagnostic testing systems for determining a hematocrit value of a sample fluid and, more particularly, to systems and methods for measuring hematocrit using impedance measurements.

### BACKGROUND OF THE INVENTION

The present disclosure relates to a biosensor system for measuring a hematocrit value associated with a blood sample. The system includes a process and system for improved determination of hematocrit, which can be applied to a test strip containing a sample fluid.

Electrochemical sensors have long been used to detect or measure the presence of substances in fluid samples. Electrochemical sensors can include a reagent mixture containing at least an electron transfer agent (also referred to as an “electron mediator”) and an analyte specific bio-catalytic protein (e.g. a particular enzyme), and one or more electrodes. Such sensors rely on electron transfer between the electron mediator and the electrode surfaces and function by measuring electrochemical redox reactions. When used in an electrochemical biosensor system or device, the electron transfer reactions are monitored via an electrical signal that correlates to the concentration of the analyte being measured in the fluid sample.

The use of such electrochemical sensors to detect analytes in bodily fluids, such as blood or blood-derived products, tears, urine, and saliva, has become important, and in some cases, vital to maintain the health of certain individuals. In the health care field, people such as diabetics, for example, must monitor a particular constituent within their bodily fluids. A number of systems are capable of testing a body fluid, such as, blood, urine, or saliva, to conveniently monitor the level of a particular fluid constituent, such as, cholesterol, proteins, and glucose. Patients suffering from diabetes, a metabolic disorder causing abnormally high glucose levels (hyperglycemia), have to monitor their blood glucose levels on a daily basis. Routine testing and control of blood glucose levels of people with diabetes can reduce their probability of long-term sequelae, such as eye, nerve, and kidney damage.

A number of systems permit people to conveniently monitor their blood glucose levels. Such systems typically include a test strip where the user applies a blood sample and a meter that “reads” the test strip to determine the glucose level in the blood sample. An exemplary electrochemical biosensor is described in U.S. Pat. No. 6,743,635 ('635 patent) which describes an electrochemical biosensor used to measure glucose level in a blood sample, and which is hereby incorporated by reference in its entirety. The electrochemical biosensor system can include a test strip and a meter. The test strip includes a sample chamber, a working electrode, a counter electrode, and fill-detect electrodes. A reagent layer can be disposed in the sample chamber, and can contain an enzyme specific for glucose, such as, glucose oxidase, or glucose dehydrogenase, and a mediator, such as, potassium ferricyanide or hexaammineruthenium chloride. When a user applies a blood sample to the sample chamber on the test strip, the reagents react with the glucose in the blood sample and the meter applies a voltage to the electrodes to cause redox reactions. The amperometric meter measures the resulting current that flows between the working and counter electrodes and calculates the glucose level based on the current measurements. Other known systems employ potentiometry and coulometry.

In some instances, electrochemical biosensors may be adversely affected by the presence of certain blood components that may undesirably affect the measurement and lead to inaccuracies in the detected signal. This inaccuracy may result in inaccurate reported blood glucose readings. As one example, the particular blood hematocrit level (i.e. the percentage of blood that is occupied by red blood cells) can in some circumstances affect a calculated and reported analyte concentration measurement.

Different levels of hematocrit, or variations in volume of red blood cells, can cause variations in glucose readings measured with disposable electrochemical test strips. Typically, a negative bias (i.e., lower calculated analyte concentration) is observed at high hematocrits, while a positive bias (i.e., higher calculated analyte concentration) is observed at low hematocrits. At high hematocrits, for example, the red blood cells may impede the diffusion of enzyme substrates and electrochemical mediators, as well as reduce the rate of chemistry dissolution because of lower plasma volume. These factors can result in a lower-than-expected glucose reading as less current is produced during the electrochemical process. Conversely, at low hematocrits fewer red blood cells and higher plasma volume reverse the phenomena associated with high hematocrit, increasing apparent glucose readings. In addition, the blood sample resistance is also hematocrit dependent, which can affect voltage or current measurements.

Several strategies have been used to reduce or avoid hematocrit-based variations on blood glucose. For example, test strips have been designed to incorporate meshes to remove red blood cells from the samples, or have included various compounds or formulations designed to increase the viscosity of red blood cell and attenuate the affect of low hematocrit on concentration determinations. Other test strips have included lysis agents, and some systems are configured to determine hemoglobin concentration in an attempt to correct for the effects of hematocrit. Further, biosensors have been configured to measure hematocrit by measuring optical variations after irradiating the blood sample with light, or measuring hematocrit based on a function of sample chamber fill time. These methods have the disadvantages of increasing the cost and complexity of test strips and may undesirably increase the time required to determine an accurate glucose measurement.

Accordingly, systems and methods for determining an accurate and efficient measurement of hematocrit are desired that overcome the drawbacks of current biosensors and improve upon existing electrochemical biosensor technologies.

### SUMMARY OF THE INVENTION

Some embodiments of this invention are directed to methods and systems for determining a hematocrit value of a blood sample. Embodiments of this invention can utilize two or more pulses of potential excitation applied at two or more frequencies. Two pulses, one applied at a “high” frequency and the other at a “low” frequency, can cause different conductive behavior when applied to a sample fluid. Impedance measurements associated with such high and low frequencies can then be used to determine a hematocrit value. Further, hematocrit may be measured before, during, or after a glucose measurement.

One embodiment consistent with the principles of this invention includes a method for determining a hematocrit value of a sample fluid described as follows. The steps include providing a sample fluid to a test strip, and applying to the test strip a first pulse at a first frequency and a second pulse at a second frequency, wherein the second frequency can be at least about 20 kHz and higher than the first frequency. The method also includes measuring a first impedance associated with the first pulse and a second impedance associated with the second pulse, and determining a hematocrit value based on the first and second impedance measurements.

A second embodiment of this invention is directed to a system for determining a hematocrit value of a sample fluid. The system includes a set of electrodes configured to apply an excitation pulse to the sample fluid. The system also includes a processor configured to measure a first impedance associated with a first pulse, wherein the first pulse can be applied to the sample fluid and the first pulse can have a first frequency. The processor can be further configured to measure a second impedance associated with a second pulse, wherein the second pulse can be applied to the sample fluid and the second pulse can have a second frequency that can be at least about 20 kHz and higher than the first frequency. The processor can be further configured to determine a hematocrit value based on the first and second impedance measurements.

A third embodiment of this invention is directed to a computer readable media, wherein the media includes a plurality of instructions configured to direct a processor to measure a first impedance associated with a first pulse, wherein the first pulse can be applied to the sample fluid and the first pulse has a first frequency. The processor can further be directed to measure a second impedance associated with a second pulse, wherein the second pulse can be applied to the sample fluid and the second pulse can have a second frequency that can be at least about 20 kHz and higher than the first frequency. Also, the processor can be directed to determine a hematocrit value based on the first and second impedance measurements.

A fourth embodiment of this invention is directed to a test strip for determining a hematocrit value and a glucose value of a blood sample. The strip can include a sample chamber configured to test a blood sample, wherein the chamber can include an aperture configured to receive the sample. The strip can also include a first set of electrodes configured to apply a first excitation to determine a hematocrit value of the sample, wherein the first set of electrodes are generally free of reagents, and a second set of electrodes configured to apply a second excitation to determine a glucose value of the sample, wherein at least one of the second set of electrodes can be located downstream of at least one of the first set of electrodes. Additionally, the strip can include a reagent layer, wherein the layer can substantially cover at least one of the second set of electrodes.

Additional embodiments consistent with principles of the invention are set forth in the detailed description which follows or may be learned by practice of methods or use of systems or articles of manufacture disclosed herein. It is understood that both the foregoing general description and the following detailed description are exemplary and explanatory only, and are not restrictive of the invention as claimed. Additionally, it is to be understood that other embodiments may be utilized and that electrical, logical, and structural changes may be made without departing form the spirit and scope of the present invention.

### BRIEF DESCRIPTION OF THE DRAWINGS

The accompanying drawings, which are incorporated in and constitute a part of this specification, illustrate several embodiments of the invention and together with the description, serve to explain the principles of the invention. In the drawings:

FIG. 1A illustrates test media associated with an exemplary meter system, according to an exemplary embodiment of the present disclosure.

FIG. 1B illustrates a test meter that can be used with test media, according to an exemplary embodiment of the present disclosure.

FIG. 1C illustrates another test meter that can be used with test media, according to an exemplary embodiment of the present disclosure.

FIG. 2A is a top plan view of a test strip, according to an exemplary embodiment of the present disclosure.

FIG. 2B is a cross-sectional view of the test strip of FIG. 2A, taken along line 2B-2B.

FIG. 3A is a top plan view of a test strip, according to another exemplary embodiment of the present disclosure.

FIG. 3B is a top plan view of a test strip, according to another exemplary embodiment of the present disclosure.

FIG. 3C is a top plan view of a test strip, according to another exemplary embodiment of the present disclosure.

FIG. 4 depicts flow chart of a method of determining a hematocrit value, according to an exemplary embodiment of the present disclosure.

### DESCRIPTION OF THE EMBODIMENTS

Reference will now be made in detail to the present embodiments of the invention, examples of which are illustrated in the accompanying drawings. Wherever possible, the same reference numbers will be used throughout the drawings to refer to the same or like parts.

In the health care field, people such as diabetics, for example, need to routinely monitor analyte levels of their bodily fluids using biosensors. A number of systems are available that allow people to test a physiological fluid (e.g. blood, urine, or saliva), to conveniently monitor the level of a particular analyte present in the fluid, such as, for example, glucose, cholesterol, ketone bodies, or specific proteins. Such systems can include a meter configured to determine the analyte concentration or display representative information to a user. In addition, such metering systems can incorporate disposable test strips configured for single-use testing of a fluid sample.

While such metering systems have been widely adopted, some are susceptible to inaccurate readings resulting from analyzing fluids of differing properties. For example, blood glucose monitoring using electrochemical techniques can be highly dependent upon hematocrit fluctuations. The present method of determining hematocrit requires measuring two or more impedance values associated with two or more pulses applied at two different frequencies. Based on these impedance values, various mathematical techniques can be used to determine a hematocrit value associated with the fluid sample. An improve analyte concentration may also be determined, based on the hematocrit value.

The present method is based on the principle that the impedance of a sample fluid is related to its water content. A sample fluid of biological cells includes water located in both intra-cellular and extra-cellular regions. The cellular membranes separating these water regions can act as an electrical insulator at certain frequencies. At certain “low” frequencies, an alternating current will generally pass through the extra-cellular region. Conversely, at certain “high” frequencies cell membranes become conductive and an alternating current can pass through both the intra- and extra-cellular regions. Impedance measurements at such low and high frequencies can then be used to determine water distribution within a biological fluid, and thus a hematocrit value.

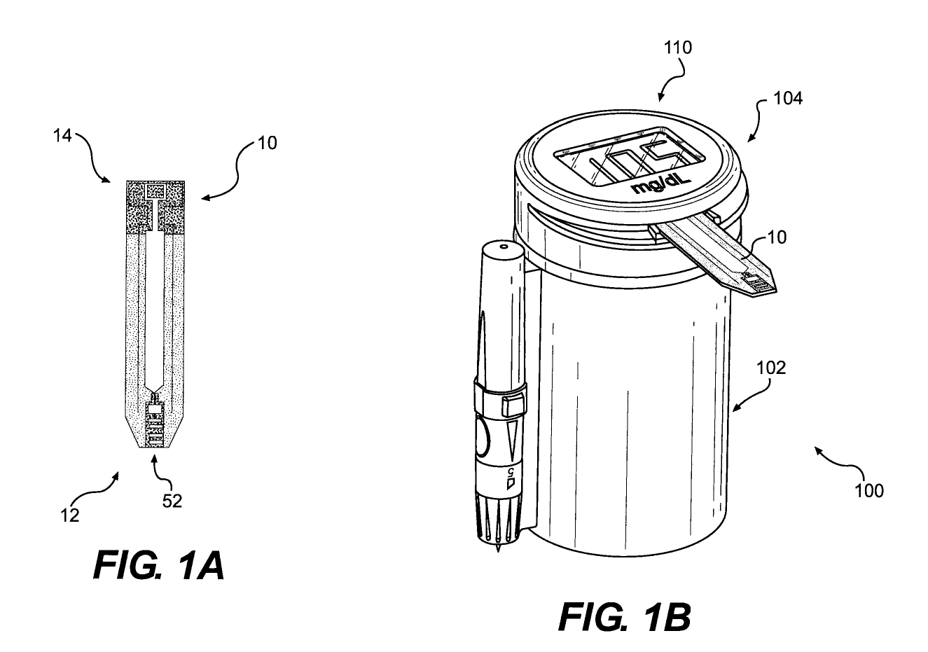

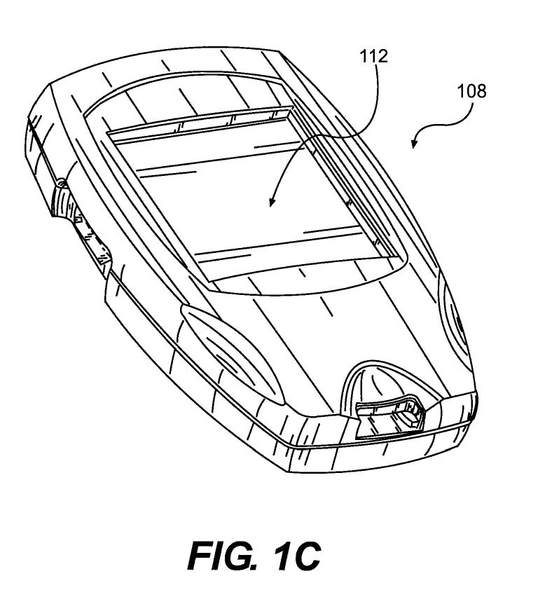

FIG. 1A illustrates a diagnostic test strip 10, according to an exemplary embodiment of the present disclosure. Test strip 10 of the present disclosure may be used with a suitable test meter 100, 108, as shown in FIGS. 1B and 1C, configured to determine hematocrit of a sample fluid, or measure the concentration of one or more analytes present in a sample fluid applied to test strip 10. As shown in FIG. 1A, test strip 10 can be generally planar and elongated in design. However, test strip 10 may be provided in any suitable form including, for example, ribbons, tubes, tabs, discs, or any other suitable form. Furthermore, test strip 10 can be configured for use with a variety of suitable testing modalities, including electrochemical tests, photochemical tests, electro-chemiluminescent tests, or any other suitable testing modality.

Test strip 10 can be in the form of a generally flat strip that extends from a proximal end 12 to a distal end 14. For purposes of this disclosure, “distal” refers to the portion of test strip 10 further from the fluid source (i.e. closer to the meter) during normal use, and “proximal” refers to the portion closer to the fluid source (e.g. a finger tip with a drop of blood for a glucose test strip) during normal use. In some embodiments, proximal end 12 of test strip 10 may include a sample chamber 52 configured to receive a fluid sample, such as, for example, a blood sample. Sample chamber 52 and test strip 10 of the present specification can be formed using materials and methods described in commonly owned U.S. Pat. No. 6,743,635, which has been incorporated herein by reference in its entirety.

Test strip 10 can be any convenient size. For example, test strip 10 can measure approximately 35 mm long (i.e., from proximal end 12 to distal end 14) and approximately 9 mm wide. Proximal end 12 can be narrower than distal end 14 in order to assist the user in locating the opening where the blood sample is to be applied. Further, test meter 100, 108 can be configured to operate with, and dimensioned to receive, test strip 10.

Test meter 100, 108 may be selected from a variety of suitable test meter types. For example, as shown in FIG. 1B, test meter 100 includes a vial 102 configured to store one or more test strips 10. The operative components of test meter 100 may be contained in a meter cap 104. Meter cap 104 may contain electrical meter components, can be packaged with test meter 100, and can be configured to close or seal vial 102. Alternatively, test meter 108 can include a monitor unit separated from storage vial, as shown in FIG. 1C.

In some embodiments, meter 100, 108 can include one or more circuits, processors, or other electrical components configured to perform one or more steps of the disclosed method of determining a hematocrit value or analyte concentration. For example, meter 100, 108 could be configured to determined a hematocrit value. In other embodiments, meter 100, 108 could be configured to determine an analyte concentration using a hematocrit value determined as described herein. Any suitable test meter may be selected to provide a diagnostic test using test strip 10 produced according to the disclosed methods.

In some embodiments, meter 100 could include a display 110 or meter 108 could include a display 112. Display 110, 112 can be configured to display a read-out to a user. For example, display 110, 112 could include a user interface or other suitable display device. In particular, display 110, 112 could be configured to display a hematocrit value, a glucose concentration, calibration data, test strip number, time, date, or other suitable output to a user. In some embodiments, display 110, 112 could be touch sensitive and configured to permit a user to select buttons displayed on display 110, 112. Other data input or output devices may also be included in meter 100, 108.

### Test Strip Configuration

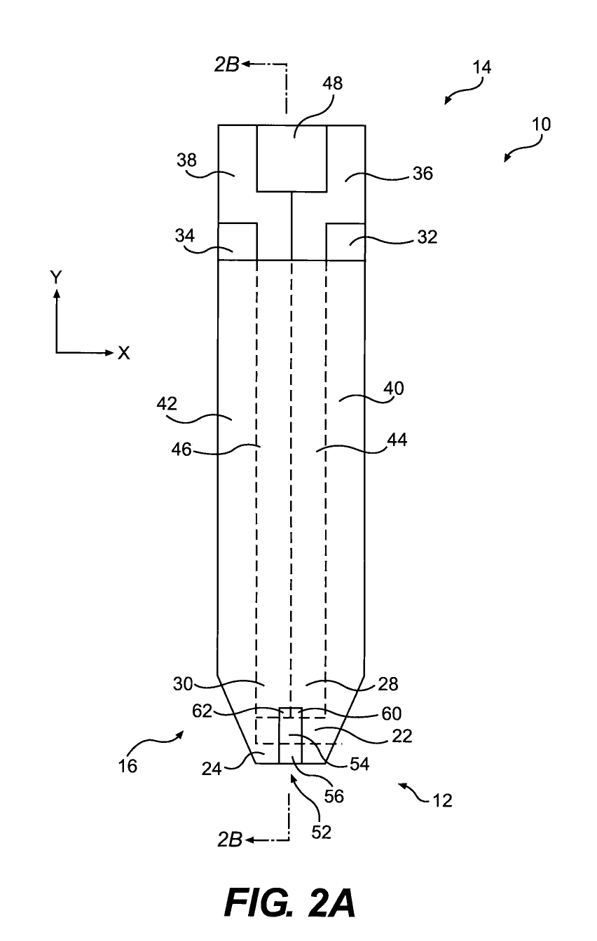

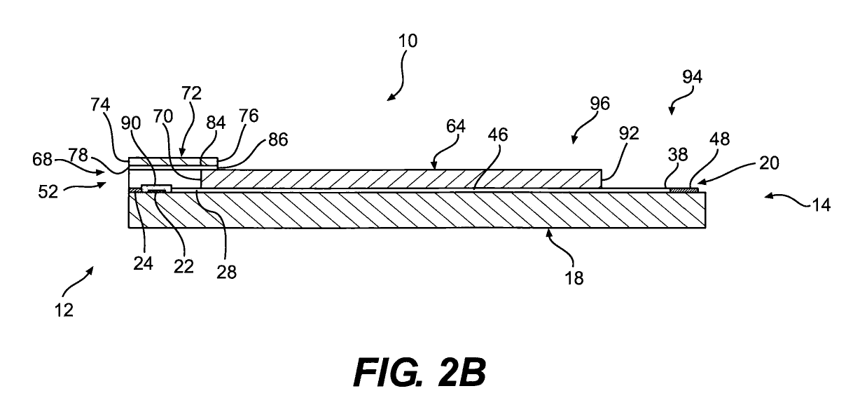

FIGS. 2A and 2B show a test strip 10, in accordance with an exemplary embodiment of the present disclosure. As shown in FIG. 2B, test strip 10 can include a generally layered construction. Working upwardly from the bottom layer, test strip 10 can include a base layer 18 extending along the entire length of test strip 10. Base layer 18 can be formed from an electrically insulating material that has a thickness sufficient to provide structural support to test strip 10. For example, base layer 18 can be a polyester material about 0.35 mm thick.

According to the illustrative embodiment, a conductive layer 20 can be disposed on base layer 18. Conductive layer 20 includes a plurality of electrodes disposed on base layer 18 near proximal end 12, a plurality of electrical contacts disposed on base layer 18 near distal end 14, and a plurality of conductive regions electrically connecting the electrodes to the electrical contacts. In the illustrative embodiment depicted in FIG. 2A, the plurality of electrodes includes a working electrode 22, a counter electrode 24, and a pair of fill-detect electrodes 28, 30. As described in detail below, the term “working electrode” refers to an electrode at which an electrochemical oxidation or reduction reaction occurs, e.g., where an analyte, typically the electron mediator, is oxidized or reduced. “Counter electrode” refers to an electrode paired with working electrode 22.

The electrical contacts at distal end 14 can correspondingly include a working electrode contact 32, a proximal electrode contact 34, and fill-detect electrode contacts 36, 38. The conductive regions can include a working electrode conductive region 40, electrically connecting working electrode 22 to working electrode contact 32, a counter electrode conductive region 42, electrically connecting counter electrode 24 to counter electrode contact 36, and fill-detect electrode conductive regions 44, 46 electrically connecting fill-detect electrodes 28, 30 to fill-detect contacts 36, 38. Further, the illustrative embodiment is depicted with conductive layer 20 including an auto-on conductor 48 disposed on base layer 18 near distal end 14.

In addition to auto-on conductor 48, the present disclosure provides test strip 10 that includes electrical contacts near distal end 14 that are resistant to scratching or abrasion. Such test strips can include conductive electrical contacts formed of two or more layers of conductive or semi-conductive material. Further, information relating to electrical contacts that are resistant to scratching or abrasion are described in co-owned U.S. patent application Ser. No. 11/458,298, which is incorporated by reference herein in its entirety.

The next layer of test strip 10 can be a dielectric spacer layer 64 disposed on conductive layer 20. Dielectric spacer layer 64 can be composed of an electrically insulating material, such as polyester. Dielectric spacer layer 64 can be about 0.100 mm thick and covers portions of working electrode 22, counter electrode 24, fill-detect electrodes 28, 30, and conductive regions 40-46, but in the illustrative embodiment does not cover electrical contacts 32-38 or auto-on conductor 48. For example, dielectric spacer layer 64 can cover substantially all of conductive layer 20 thereon, from a line just proximal of contacts 32 and 34 all the way to proximal end 12, except for sample chamber 52 extending from proximal end 12. In this way, sample chamber 52 can define an exposed portion 54 of working electrode 22, an exposed portion 56 of counter electrode 24, and exposed portions 60, 62 of fill-detect electrodes 28, 30.

In some embodiments, sample chamber 52 can include a first opening 68 at proximal end 12 of test strip 10, and a second opening 86 for venting sample chamber 52. Further, sample chamber 52 may be dimensioned or configured to permit, by capillary action, a blood sample to enter through first opening 68 and remain within sample chamber 52. For example, sample chamber 52 can be dimensioned to receive about 1 micro-liter or less. For example, first sample chamber 52 can have a length (i.e., from proximal end 12 to distal end 70) of about 0.140 inches, a width of about 0.060 inches, and a height (which can be substantially defined by the thickness of dielectric spacer layer 64) of about 0.005 inches. Other dimensions could be used, however.

A cover 72, having a proximal end 74 and a distal end 76, can be attached to dielectric spacer layer 64 via an adhesive layer 78. Cover 72 can be composed of an electrically insulating material, such as polyester, and can have a thickness of about 0.1 mm. Additionally, the cover 72 can be transparent. Adhesive layer 78 can include a polyacrylic or other adhesive and have a thickness of about 0.013 mm. A break 84 in adhesive layer 78 can extend from distal end 70 of first sample chamber 52 to an opening 86, wherein opening 86 can be configured to vent sample chamber 52 to permit a fluid sample to flow into sample chamber 52. Alternatively, cover 72 can include a hole (not shown) configured to vent sample chamber 52. It is also contemplated that various materials, surface coatings (e.g. hydrophilic or hydrophobic), or other structure protrusions or indentations at proximal end 12 may be used to form a suitable sample reservoir.

As shown in FIG. 2B, a reagent layer 90 can be disposed in sample chamber 52. In some embodiments, reagent layer 90 can include one or more chemical constituents to enable the level of glucose in the blood sample to be determined electrochemically. Reagent layer 90 may include an enzyme specific for glucose, such as glucose oxidase or glucose dehydrogenase, and a mediator, such as potassium ferricyanide or hexaammineruthenium chloride. In other embodiments, other reagents or other mediators can be used to facilitate detection of glucose and other analytes contained in blood or other physiological fluids. In addition, reagent layer 90 may include other components, buffering materials (e.g., potassium phosphate), polymeric binders (e.g., hydroxypropyl-methyl-cellulose, sodium alginate, microcrystalline cellulose, polyethylene oxide, hydroxyethylcellulose, or polyvinyl alcohol), and surfactants (e.g., Triton X-100 or Surfynol 485). For example, an exemplary formulation contains 50-250 mM potassium phosphate at pH 6.75-7.50, 150-190 mM hexaammineruthenium chloride, 3500-5000 U/mL PQQ-dependent glucose dehydrogenase, 0.5-2.0% polyethylene oxide, 0.025-0.20% NATROSOL 250M (hydroxyethylcellulose), 0.675-2.5% Avicel (microcrystalline cellulose), 0.05-0.25% TRITON-X (surfactant) and 2.5-5.0% trehalose.

In some embodiments, various constituents may be added to reagent layer 90 to at least partially reduce unwanted bias of an analyte measurement. For example, various polymers, molecules, or compounds may be added to reagent layer 90 to reduce cell migration and hence may increase the accuracy of a measurement based on an electrochemical reaction. Also, one or more conductive components may be coated with a surface layer (not shown) to at least partially restrict cell migration onto the one or more conductive components. These and other techniques known in the art may be used to reduce unwanted signal bias.

Although FIGS. 2A and 2B illustrate an illustrative embodiment of test strip 10, other configurations, chemical compositions, and electrode arrangements could be used. For example, fill-detect electrode 30 can function with working electrode 22 to perform a fill-detect feature, as previously described. Other configurations of electrodes on test strip 10 are possible, such as, for example, a single fill-detect electrode, multiple fill-detect electrodes aligned in the y-axis (as opposed to the x-axis as shown in FIG. 2A), or multiple working electrodes.

In some embodiments, electrodes may be configured to permit a hematocrit measurement, as described in detail below. Further, hematocrit may be measured before a glucose measurement. Measuring hematocrit before glucose may reduce diffusion of a glucose reagent during a hematocrit measurement.

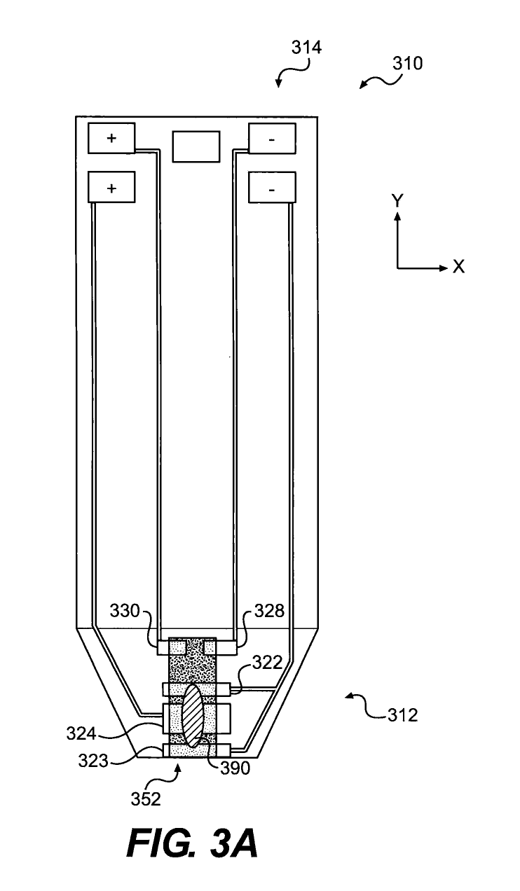

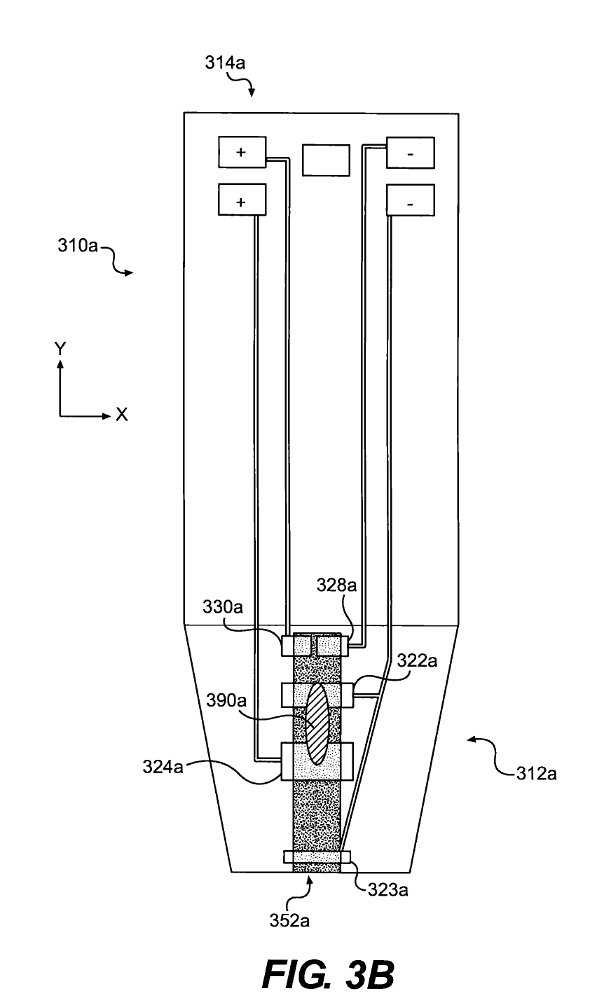

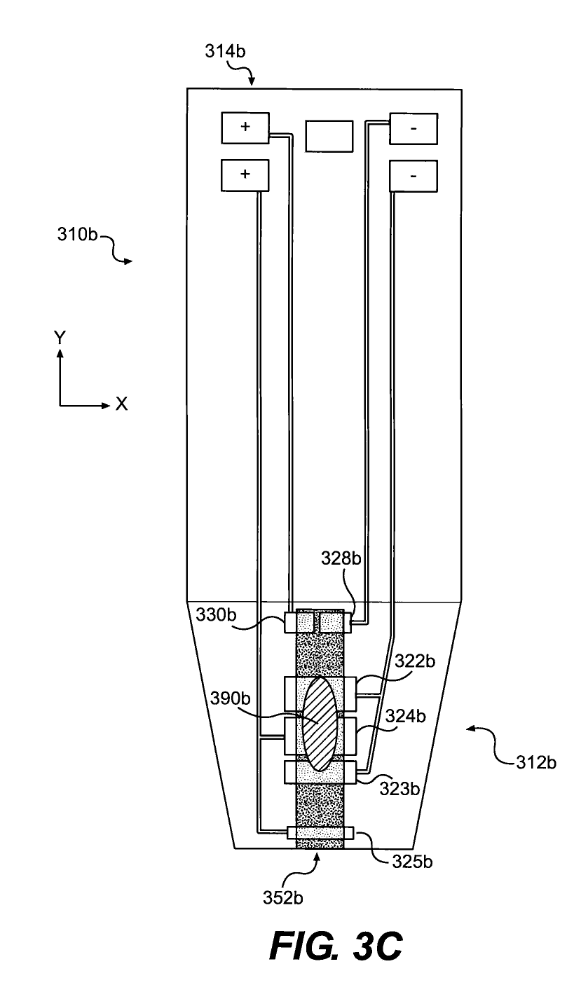

FIG. 3A shows a test strip 310, according to an exemplary embodiment of the present disclosure. Similar to test strip 10 shown in FIG. 2A, test strip 310 can include a proximal end 312 and a distal end 314. Distal end 314 can include a plurality of electrical contacts configured to permit operation of test strip 310 with meter 100, 108. Proximal end 312 can include a plurality of electrodes configured for hematocrit or glucose measurement. Specifically, proximal end 312 can include one or more working electrodes 322, 323, a counter electrode 324, and a pair of fill-detect electrodes 328, 330. A reagent layer 390 can cover one or more regions of the electrodes housed in a sample chamber 352.

In some instances, hematocrit may be measured before a glucose measurement is taken. To reduce the influence of reagents, hematocrit could be measured before a blood sample substantially mixes with a reagent layer. For example, hematocrit may be measured across two electrodes exposed to limited levels of one or more reagents. As shown in FIG. 3B, a proximal working electrode 323a can be positioned in sample chamber 352a proximal to counter electrode 324a. In effect, proximal working electrode 323a is located upstream of counter electrode 324a as blood will pass over proximal working electrode 323a before reaching counter electrode 324a. The region between proximal working electrode 323a and counter electrode 324a can be generally free of reagent layer 390a.

In operation, as a blood sample travels up sample chamber 352a from an aperture at a proximal location (i.e., at the bottom of FIG. 3B), the blood first passes proximal working electrode 323a and then reaches counter electrode 324a located downstream. If reagent layer 390a is generally located distally to counter electrode 324a, hematocrit could be measured between this electrode and proximal working electrode 323a without substantially mixing the blood and reagents. Following continued downstream (i.e., distal) movement up chamber 352a, the blood may more fully mix with layer 390a. When the blood reaches fill-detect electrodes 328a, 330a, an electrochemical glucose measurement could be made between working electrode 322a and counter electrode 324a.

FIG. 3C shows test strip 310b, illustrating another exemplary electrode configuration for measuring hematocrit before glucose. In this embodiment, test strip 310b includes a proximal counter electrode 325b, located generally upstream of reagent layer 390b in chamber 352b. As described above for FIG. 3B, this upstream electrode 325b may be used to determine hematocrit before the blood sample substantially mixes with reagents 390b. As such, hematocrit may be determined using electrodes 323b and 325b, while glucose may be determined using electrodes 322b and 324b. Other electrode configurations, whereby some electrodes are substantially more exposed to reagents than other electrodes, are also contemplated by the current disclosure. Also, the polarities of the various electrodes described herein may be reversed or modified by one of ordinary skill in the art.

### Test Strip and Meter Operation

As previously described, test strip 10, 310 can be configured for placement within meter 100, 108, or similar device configured to determine the concentration of an analyte. Meter 100, 108 can include electrical components, circuitry, or processors configured to perform various operations to determine analyte concentration based on electrochemical techniques. For example, the metering system, such as meter 100, 108 and associated test strip 10, 310, may be configured to determine a hematocrit value or a glucose concentration of a blood sample. In some embodiments, systems and methods of the present disclosure permit determination of blood glucose levels generally unaffected by blood constituents, hematocrit levels, and temperature.

In operation, the battery-powered meter 100, 108 may stay in a low-power sleep mode when not in use. When test strip 10, 310 is inserted into meter 100, 108, one or more electrical contacts at distal end 14, 314 of test strip 10, 310 could form electrical connections with one or more corresponding electrical contacts in meter 100, 108. These electrical contacts may bridge electrical contacts in meter 100, 108, causing a current to flow through a portion of the electrical contacts. Such a current flow can cause meter 100, 108 to “wake-up” and enter an active mode.

Meter 100, 108 can read encoded information provided by the electrical contacts at distal end 14, 314. Specifically, the electrical contacts can be configured to store information, as described in U.S. patent application Ser. No. 11/458,298. In particular, an individual test strip 10, 310 can include an embedded code containing data associated with a lot of test strips, or data particular to that individual strip. The embedded information can represent data readable by meter 100, 108. For example, a microprocessor associated with meter 100, 108 could access and utilize a specific set of stored calibration data specific to an individual test strip 10, 310 or a manufactured lot test strips 10. Individual test strips 10, 310 may be calibrated using standard solutions, and associated calibration data could be applied to test strips 10, 310 of the same or similar lots of manufactured test strips 10, 310.

In some embodiments, “lot specific” calibration information can be encoded on a code chip accompanying a vial of strips, or coded directly onto one or more test strips manufactured in a common lot of test strips. Lot calibration can include any suitable process for calibrating test strip 10, 310 or meter 100, 108. For example, calibration can include applying at the factory a standard fluid to one or more test strips from a manufacturing lot, wherein the standard fluid can be a solution of known glucose concentration, hematocrit, temperature, or any other appropriate parameter associated with the solution. Following application of the standard fluid, one or more pulses can be applied to test strip 10, 310, as described below. Calibration data may then be determined by correlating various measurements to be determined by the meter 100, 108 during use by the patient with one or more parameters associated with the standard fluid. For example, a measured current may be correlated with a glucose concentration, or a voltage correlated with hematocrit. Such calibration data, that can vary from lot to lot with the performance of the test strips, may then be stored on test strip 10, 310 or meter 100, 108, and used to determine analyte concentration of an analyte sample, as described below.

Test strip 10, 310 can be tested at any suitable stage during a manufacturing process. Also, a test card (not shown) could be tested during any suitable stage of a manufacturing process, as described in co-owned U.S. patent application Ser. No. 11/504,710 which is incorporated by reference herein in its entirety. Such testing of the test strip or the test card can permit determination or encoding of calibration data at any suitable stage during a manufacturing process. For example, calibration data associated with methods of the present disclosure can be encoded during the manufacturing process.

In operation meter 100, 108 can be configured to identify a particular test to be performed or provide a confirmation of proper operating status. Also, calibration data pertaining to the strip lot, for either the analyte test or other suitable test, could be otherwise encoded or represented, as described above. For example, meter 100, 108 can identify the inserted strip as either the test strip or a check strip (not shown) based on the particular code information.

If meter 100, 108 detects test strip 10, it may perform a test strip sequence. The test strip sequence may confirm proper functioning of one or more components of test strip 10, 310. For example, meter 100, 108 could validate the function of working electrode 22, 322 counter electrode 24, 324 and, if included, the fill-detect electrodes, by confirming that there are no low-impedance paths between any of these electrodes. If the electrodes are valid, meter 100, 108 could provide an indication to the user that a sample may be applied to test strip 10, 310.

If meter 100, 108 detects a check strip, it may perform a check strip sequence. The system may also include a check strip configured to confirm that the instrument is electrically calibrated and functioning properly. The user may insert the check strip into meter 100, 108. Meter 100, 108 may then receive a signal from the check strip to determine if meter 100, 108 is operating within an acceptable range.

In other embodiments, the test strip or the meter may be configured to perform a calibration process based on a standard fluid, also termed a control solution. The control solution may be used to periodically test one or more functions of the system. For example, a control solution may include a solution of a known property, such as, hematocrit or analyte concentration, and an electrical measurement of the solution may be performed by meter 100, 108. Upon detecting the presence of a control solution, the meter can perform an operational check of test strip 10, 310 functionality to verify measurement integrity. For example, the read-out of the meter may be compared to a known hematocrit or glucose value of the solution to confirm that meter 100, 108 and test strip 10, 310 are functioning to an appropriate accuracy. In addition, any data associated with a measurement of a control solution may be processed, stored or displayed using meter 100, 108 differently to any data associated with a hematocrit or glucose measurement. Such different treatment of data associated with the control solution may permit the meter, or user, to distinguish a hematocrit or glucose measurement, or may permit exclusion of any control measurements when conducting any mathematical analysis of hematocrit or glucose measurements.

### Hematocrit Determination

Meter 100, 108 can be configured to apply a signal to test strip 10, 310 to determine a hematocrit value of a fluid contacting the test strip. The meter can also be configured to apply a signal to the test strip to determine a concentration of an analyte contained in a fluid contacting the strip, based on the hematocrit value. In some cases, the signal can be applied following a determination that sample chamber 52, 352 of test strip 10, 310 contains a sufficient quantity of fluid sample. To determine the presence of sufficient fluid, meter 100, 108 can apply a detect voltage between any suitably configured electrodes, such as, for example, fill-detect electrodes. The detect voltage can detect the presence of sufficient quantity of fluid (e.g. blood) within the sample chamber by detecting a current flow between the fill-detect electrodes. If required, to determine that the fluid sample has traversed reagent layer 90, 390 and mixed with the chemical constituents in the reagent layer, meter 100, 108 may apply a fill-detect voltage to the one or more fill-detect electrodes and measure any resulting current. If the resulting current reaches a sufficient level within a predetermined period of time, the meter can indicate to a user that adequate sample is present. Meter 100, 108 can also be programmed to wait for a predetermined period of time after initially detecting the blood sample to allow the blood sample to react with the reagent layer. Alternatively, the meter can be configured to immediately begin taking readings in sequence.

Meter 100, 108 can be configured to apply various signals to test strip 10, 310. For example, an exemplary fluid measurement sequence could include amperometry, wherein an assay voltage is applied between the working and counter electrodes of the strip. The magnitude of the assay voltage can include any suitable voltage, and could be approximately equal to the redox potential of constituents of the reagent layer. Following application of an assay voltage, also termed potential excitation, meter 100, 108 could be configured to measure one or more current values between the working and counter electrodes. Such a measured current can be mathematically related to the concentration of analyte in the fluid sample, such as, for example, glucose concentration in a blood sample.

For example, one or more constituents of reagent layer 90, 390 may react with glucose present in a blood sample such that glucose concentration may be determined using electrochemical techniques. Suitable enzymes of reagent layer 90, 390 (e.g. glucose oxidase or glucose dehydrogenase) could react with blood glucose. In some instances, glucose can be oxidized to form gluconic acid while the enzyme, or its associated co-enzyme, can be reduced. The reduced enzyme can then reduce a mediator, such as, for example, potassium ferricyanide. Voltage applied to working electrode 22, 322 may oxidize the ferrocyanide to form ferricyanide, thereby generating a current proportional to the glucose concentration of the blood sample.

As previously discussed, measurements of analyte concentration using a biosensor may be inaccurate due to unwanted effects of various blood components. For example, the hematocrit level of blood can erroneously affect a measurement of analyte concentration. In order to reduce inaccuracies associated with a determination of analyte concentration, it may be advantageous to determine a hematocrit value of the blood sample.

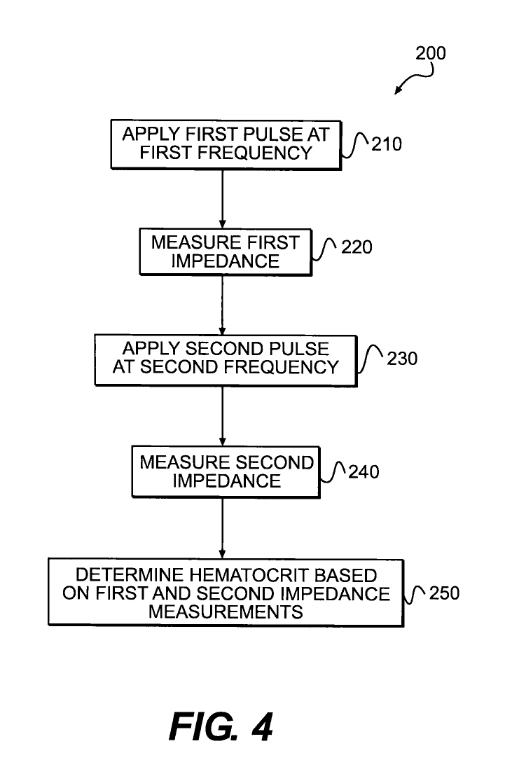

FIG. 4 depicts a method 200 for determining a hematocrit value of a sample fluid, according to an exemplary embodiment of the present disclosure. For example, the fluid sample may include blood and may be contained within test strip 10, 310. As described above, meter 100, 108 can be configured to supply a potential excitation to one or more electrodes within test strip 10, 310. Based on the application of the potential to the test strip, a hematocrit value associated with the fluid sample may be determined. In other embodiments, based on the hematocrit value, a concentration of an analyte contained within the fluid sample may also be determined.

Initially, a first pulse of potential excitation can be applied to a fluid sample (Step 210). The first pulse may include an alternating waveform or a waveform including an alternating waveform component. The first pulse may include a DC offset, an offset of generally known waveform, or a generally constant voltage. In some embodiments, the frequency of the first pulse may be at least about 20 kHz. In other embodiments, the frequency of the first pulse may be at least about 50 kHz, 100 kHz, 250 kHz, 1 MHz, 10 MHz, or 100 MHz. It is also contemplated that, in certain situations, a first frequency may be at least about 10 kHz. As described above, the frequency of the first pulse may be low enough such that the resulting alternating current predominantly passes through the extra-cellular region of the biological fluid.

Meter 100, 108 can also be configured to measure a first impedance associated with the first pulse (Step 220). This first impedance value may be measured during the application of the first pulse, wherein the first pulse is applied for a limited time. In some embodiments, this time could be less than about 10 seconds, less than about 1 second, less than about 100 milliseconds, or less than about 100 microseconds. One or more impedance values, of similar or differing durations, may be measured.

Following, a second pulse of potential excitation can be applied to a fluid sample (Step 230). The second pulse may be generally similar to the first pulse, and may also include an alternating waveform or a waveform including an alternating waveform component. The frequency of the second pulse can be higher than the frequency of the first pulse. In some embodiments, the frequency of the second pulse may be higher than the frequency of the first pulse by a multiple of 2, 5, 10, 20, 50, 100, 200, 500, or 1,000. For example, the first pulse may include a waveform with a frequency of about 10 kHz and the second pulse may include a waveform with a frequency of about 50 kHz or about 250 kHz. In other examples, the first and second pulses may have frequencies of 50 kHz and 1 MHz, respectively. As described above, the frequency of the second pulse may be high enough such that the resulting alternating current passes generally through both the intra-cellular and extra-cellular regions of the biological fluid.

The meter can also be configured to measure a second impedance associated with the second pulse (Step 240). This second impedance value may be measured during the application of the second pulse, wherein the second pulse is applied for a limited time, as similarly described above for the first pulse.

In some embodiments, the order of applying the high and low frequency pulses to the sample fluid may be reversed. For example, the first pulse may include a high frequency and the second pulse may include a low frequency. One of ordinary skill will also appreciate that a combination of high and low pulses may be applied. Further, pulses of variable frequency may also be utilized. In general, two or more pulses of various waveform, frequency, amplitude, or duration may be utilized by the present method. Also, a single pulse of variable frequency, or train of pulses at various frequencies, may also be used.

Generally, impedance values can be measured using a sample prior to mixing with glucose reagents. In some instances, some reagents can be combined with a sample, while in other instances the sample and reagent can be well combined before measuring impedance. In contrast to prior art methods for determining glucose concentration using hematocrit, such as described in U.S. Application Publication No. 2004/0256248, the present method does not require mixing reagents and sample to ensure an accurate hematocrit measurement.

In some embodiments, an impedance measurement can include a measure of resistance or reactance. Other types of suitable measurement are also known to those skilled in the art, such as, for example, phase angle. Such measurements can be made simultaneously or sequentially. Also, one or more impedance measurements may be obtained for each pulse. For example, a reactive component may be measured at a first frequency while a resistive component may be measured at a second frequency. Values representing an average, mean, standard deviation, slope, initial, or final impedance may also be utilized by the present method. In some situations, various impedance measurements may be stored for later use, as described above.

Next, a hematocrit value may be determined based on the first and second impedance measurements (Step 250). Correlative and error analysis techniques may be used to determine a relationship between hematocrit and impedance. Linear and multiple regression analysis may be performed to develop an equation wherein hematocrit is dependent upon one or more impedance measurements. For example, hematocrit can be determined using the following equation:

$$HCT = A ln ( {X}_{1 MHz} ) + B ln ( \frac{{R}_{50 kHz}}{{R}_{1 MHz}} ) + C$$

Variables A, B, and C can be any positive or negative real values, HCT can be hematocrit, X_{1 MHz} can be reactance at 1 MHz, and R_{50 kHz} and R_{1 MHz} can be resistance at 50 kHz and 1 MHz respectively. Various mathematical techniques can be used to determine variables A, B, and C. As such, HCT can be determined by measuring X_{1 MHz}, R_{50 kHz}, and R_{1 MHz}. This and various other equations are contemplated by the present method. For example, resistance or reactance may be determined for frequencies of 10, 50, or 250 kHz. Also, a relationship between HCT and impedance measurements could be described by a lookup table, an array, or any other suitable data structure.

Following determination of a hematocrit value, the hematocrit value may be displayed as described above using the meter. In other instances, the hematocrit value may be stored in memory or used to in another calculation or procedure associated with the sample fluid. For example, analyte concentration may be determined based on the hematocrit value, as described above. Some techniques for determining analyte concentration are also described in co-owned U.S. patent application Ser. No. 12/179,970, filed Jul. 25, 2008 and U.S. patent application Ser. No. 12/115,804, filed May 6, 2008, both of which are incorporated by reference herein in their entirety. In another example, the hematocrit value could be used to determine a calibration curve, as described above.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for determining a hematocrit value of a sample fluid, comprising:
providing a sample fluid to a test strip;
applying to the test strip a first pulse at a first frequency and a second pulse at a second frequency, wherein the second frequency is at least about 20 kHz and higher than the first frequency;
measuring a first impedance associated with the first pulse and a second impedance associated with the second pulse; and
determining a hematocrit value based on the first and second impedance measurements.

2. The method of claim 1, wherein the first frequency is greater than at least one of 10 kHz, 50 kHz, 100 kHz, and 1 MHz.

3. The method of claim 1, wherein the second frequency is higher than the first frequency by a multiple of at least one of 2, 5, 10, 20, 50, and 100.

4. The method of claim 1, wherein impedance includes at least one of a resistive component, a reactive component, and a phase angle.

5. The method of claim 1, further comprising:
determining a concentration of an analyte within the sample fluid based in part on the hematocrit value.

6. The method of claim 5, wherein the analyte is glucose.

7. The method of claim 6, wherein the sample fluid includes an enzyme of at least one of glucose oxidase and glucose dehydrogenase and a mediator of at least one of potassium ferricyanide and hexaammineruthenium chloride.

8. The method of claim 1, further comprising:
determining a calibration curve based on one or more hematocrit values.

9. The method of claim 1, wherein the first pulse is applied following the application of the second pulse.

10. A system for determining a hematocrit value of a sample fluid, comprising:
a set of electrodes configured to apply an excitation pulse to the sample fluid;
a processor configured to:
measure a first impedance associated with a first pulse, wherein the first pulse is applied to a sample fluid and the first pulse has a first frequency;
measure a second impedance associated with a second pulse, wherein the second pulse is applied to the sample fluid, and has a second frequency that is at least about 20 kHz and higher than the first frequency; and
determine a hematocrit value based on the first and second impedance measurements.

11. The system of claim 10, wherein the first frequency is greater than at least one of 10 kHz, 50 kHz, 100 kHz, and 1 MHz.

12. The system of claim 10, wherein the second frequency is higher than the first frequency by a multiple of at least one of 2, 5, 10, 20, 50, and 100.

13. The system of claim 10, wherein impedance includes at least one of a resistive component, a reactive component, and a phase angle.

14. The system of claim 10, wherein the processor is further configured to determine a concentration of an analyte within the sample fluid based in part on the hematocrit value.

15. The system of claim 14, wherein the analyte is glucose.

16. The system of claim 15, wherein the sample fluid includes an enzyme of at least one of glucose oxidase and glucose dehydrogenase and a mediator of at least one of potassium ferricyanide and hexaammineruthenium chloride.

17. The system of claim 10, wherein the processor is further configured to determine a calibration curve based on one or more hematocrit values.

18. The system of claim 10, further including a display configured to show the hematocrit value.

19. The system of claim 10, wherein the processor is further configured to apply the first pulse following application of the second pulse.

20. A computer readable media, wherein the media comprises a plurality of instructions configured to direct a processor to:
measure a first impedance associated with a first pulse, wherein the first pulse is applied to a sample fluid and the first pulse has a first frequency;
measure a second impedance associated with a second pulse, wherein the second pulse is applied to the sample fluid, and has a second frequency that is at least about 20 kHz and higher than the first frequency; and
determine a hematocrit value based on the first and second impedance measurements.

21. The computer readable media of claim 20, wherein the first frequency is greater than at least one of 10 kHz, 50 kHz, 100 kHz, and 1 MHz.

22. The computer readable media of claim 20, wherein the second frequency is higher than the first frequency by a multiple of at least one of 2, 5, 10, 20, 50, and 100.

23. The computer readable media of claim 20, wherein impedance includes at least one of a resistive component, a reactive component, and a phase angle.

24. The computer readable media of claim 20, wherein the instructions further direct the processor to determine a concentration of an analyte within the sample fluid based in part on the hematocrit value.

25. The computer readable media of claim 24, wherein the analyte is glucose.

26. The computer readable media of claim 25, wherein the sample fluid includes an enzyme of at least one of glucose oxidase and glucose dehydrogenase and a mediator of at least one of potassium ferricyanide and hexaammineruthenium chloride.

27. The computer readable media of claim 20, wherein the instructions further direct the processor to determine a calibration curve based on one or more hematocrit values.

28. The computer readable media of claim 20, wherein the instructions further direct the processor to output a signal representing the hematocrit value to a display.

29. The computer readable media of claim 20, wherein the instructions further direct the processor to apply the first pulse following application of the second pulse.

30. A test strip for determining a hematocrit value and a glucose value of a blood sample, comprising:
a sample chamber configured to test a blood sample, wherein the chamber includes an aperture configured to receive the sample;
a first set of electrodes configured to apply a first excitation to determine a hematocrit value of the sample, wherein the first set of electrodes are generally free of reagents;
a second set of electrodes configured to apply a second excitation to determine a glucose value of the sample, wherein at least one of the second set of electrodes is located downstream of at least one of the first set of electrodes;
a reagent layer, wherein the layer substantially covers at least one of the second set of electrodes.

31. The test strip of claim 30, wherein at least one of the first set of electrodes and at least one of the second set of electrodes is the same electrode.

32. The test strip of claim 30, wherein the reagent layer includes an enzyme of at least one of glucose oxidase and glucose dehydrogenase and a mediator of at least one of potassium ferricyanide and hexaammineruthenium chloride.
